(19) United States
(12) Patent Application Publication
(10) Pub. No.: US 2021/0383927 A1
(43) Pub. Date: Dec. 9, 2021

(54) DOMAIN-TRANSFERRED HEALTH-RELATED PREDICTIVE DATA ANALYSIS

(71) Applicant: Optum, Inc., Minnetonka, MN (US)

(72) Inventors: Paul J. Godden, London (GB); Olusola Omosaiye, Cary, NC (US); Sarah McCandless, Raleigh, NC (US); Gregory J. Boss, Saginaw, MI (US)

(21) Appl. No.: 16/895,424

(22) Filed: Jun. 8, 2020

(51) Int. Cl. G16H 50/30; G16H 50/20; G16H 50/70; G16H 10/60; G06N 5/04

(52) U.S. Cl. CPC G16H 50/30; G16H 50/20; G06N 5/04; G16H 10/60; G16H 50/70

(57) ABSTRACT

There is a need for more effective and efficient health-related predictive data analysis. This need can be addressed by, for example, solutions for performing domain-transferred health-related predictive data analysis. In one example, a method includes identifying an initial risk scoring model, generating a cross-domain mapping of the initial risk scoring model that maps initial risk categories of the initial risk scoring model to inferred risk categories, generating a weighted risk category value for each inferred risk category, generating a health-related risk prediction based on each weighted risk category value, and performing prediction-based actions based on the health-related risk prediction.

### BACKGROUND

Various embodiments of the present invention address technical challenges related to performing health-related predictive data analysis. Various embodiments of the present invention address the shortcomings of existing health-related predictive data analysis systems and disclose various techniques for efficiently and reliably performing health-related predictive data analysis.

### BRIEF SUMMARY

In general, embodiments of the present invention provide methods, apparatus, systems, computing devices, computing entities, and/or the like for performing health-related predictive data analysis. Certain embodiments of the present invention utilize systems, methods, and computer program products that perform health-related predictive data analysis by utilizing at least one of cross-domain mappings, inferred risk category, and per-category weight values for inferred risk categories. Examples of health-related predictive data analysis tasks include genetic predictive data analysis tasks, polygenic predictive data analysis tasks, medical predictive data analysis tasks, behavioral predictive data analysis tasks, and/or medical predictive data analysis tasks.

In accordance with one aspect, a method is provided. In one embodiment, the method comprises: identifying an initial risk scoring model, wherein the initial risk scoring model is associated with a plurality of initial risk categories; generating a cross-domain mapping of the initial risk scoring model, wherein: (i) the cross-domain mapping maps each initial risk category of the plurality of initial risk categories to an inferred risk category of a plurality of inferred risk categories, and (ii) each inferred risk category of the plurality of inferred risk categories is associated with one or more observed input variables for a target individual; for each inferred risk category of the plurality of inferred risk categories: determining an inferred risk category value for the inferred risk category based on the one or more observed input variables for the inferred risk category, determining a per-category weight value for the inferred risk category value, and determining a weighted risk category value for the inferred risk category based on the inferred risk category value for the inferred risk category and the per-category weight value for the inferred risk category; processing each weighted risk category value for an inferred risk category of the plurality of inferred risk categories using the initial risk scoring model and in accordance with the cross-domain mapping in order to generate a health-related risk prediction for the target individual with respect to a target condition; and performing one or more prediction-based actions based on the health-related risk prediction.

In accordance with another aspect, a computer program product is provided. The computer program product may comprise at least one computer-readable storage medium having computer-readable program code portions stored therein, the computer-readable program code portions comprising executable portions configured to: identify an initial risk scoring model, wherein the initial risk scoring model is associated with a plurality of initial risk categories; generate a cross-domain mapping of the initial risk scoring model, wherein: (i) the cross-domain mapping maps each initial risk category of the plurality of initial risk categories to an inferred risk category of a plurality of inferred risk categories, and (ii) each inferred risk category of the plurality of inferred risk categories is associated with one or more observed input variables for a target individual; for each inferred risk category of the plurality of inferred risk categories: determine an inferred risk category value for the inferred risk category based on the one or more observed input variables for the inferred risk category, determine a per-category weight value for the inferred risk category value, and determine a weighted risk category value for the inferred risk category based on the inferred risk category value for the inferred risk category and the per-category weight value for the inferred risk category; process each weighted risk category value for an inferred risk category of the plurality of inferred risk categories using the initial risk scoring model and in accordance with the cross-domain mapping in order to generate a health-related risk prediction for the target individual with respect to a target condition; and perform one or more prediction-based actions based on the health-related risk prediction.

In accordance with yet another aspect, an apparatus comprising at least one processor and at least one memory including computer program code is provided. In one embodiment, the at least one memory and the computer program code may be configured to, with the processor, cause the apparatus to: identify an initial risk scoring model, wherein the initial risk scoring model is associated with a plurality of initial risk categories; generate a cross-domain mapping of the initial risk scoring model, wherein: (i) the cross-domain mapping maps each initial risk category of the plurality of initial risk categories to an inferred risk category of a plurality of inferred risk categories, and (ii) each inferred risk category of the plurality of inferred risk categories is associated with one or more observed input variables for a target individual; for each inferred risk category of the plurality of inferred risk categories: determine an inferred risk category value for the inferred risk category based on the one or more observed input variables for the inferred risk category, determine a per-category weight value for the inferred risk category value, and determine a weighted risk category value for the inferred risk category based on the inferred risk category value for the inferred risk category and the per-category weight value for the inferred risk category; process each weighted risk category value for an inferred risk category of the plurality of inferred risk categories using the initial risk scoring model and in accordance with the cross-domain mapping in order to generate a health-related risk prediction for the target individual with respect to a target condition; and perform one or more prediction-based actions based on the health-related risk prediction.

### BRIEF DESCRIPTION OF THE DRAWINGS

Having thus described the invention in general terms, reference will now be made to the accompanying drawings, which are not necessarily drawn to scale, and wherein:

FIG. 1 provides an exemplary overview of an architecture that can be used to practice embodiments of the present invention.

FIG. 2 provides an example predictive data analysis computing entity in accordance with some embodiments discussed herein.

FIG. 3 provides an example external computing entity in accordance with some embodiments discussed herein.

FIG. 4 is a flowchart diagram of an example process for performing health-related predictive data analysis for a target individual in relation to a target condition in accordance with some embodiments discussed herein.

FIG. 5 is a data flow diagram of an example process for generating a cross-domain mapping for an initial risk scoring model in accordance with some embodiments discussed herein.

FIG. 6 is a data flow diagram of an example process for generating an updated health-related risk prediction in accordance with some embodiments discussed herein.

FIG. 7 provides an operational example of a predictive output user interface in accordance with some embodiments discussed herein.

### DETAILED DESCRIPTION

Various embodiments of the present invention now will be described more fully hereinafter with reference to the accompanying drawings, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. The term “or” is used herein in both the alternative and conjunctive sense, unless otherwise indicated. The terms “illustrative” and “exemplary” are used to be examples with no indication of quality level. Like numbers refer to like elements throughout. Moreover, while certain embodiments of the present invention are described with reference to predictive data analysis, one of ordinary skill in the art will recognize that the disclosed concepts can be used to perform other types of data analysis.

### I. OVERVIEW

Various embodiments of the present invention address technical challenges related to improving computational efficiency and/or operational reliability of performing health-related predictive data analysis. Health-related predictive data analysis systems face substantial challenges because they are tasked with integrating predictive insights related to physiological diversity across the human population (e.g., the genetic diversity of human genome across humans). Because of the noted challenges, various existing predictive data analysis solutions are either highly ineffective and/or too computationally costly. Meanwhile, many other areas of predictive risk scoring (e.g., financial risk scoring, such as credit risk scoring) have models that perform more efficiently and/or more effectively in their respective domains relative to the performance of various existing health-related predictive data analysis solutions in the polygenic risk scoring domain.

To address the noted concerns related to computational efficiency and/or operational reliability of performing health-related predictive data analysis, various embodiments of the present invention introduce innovative techniques for transferring medical/polygenic input data into variables for non-polygenic predictive models by mapping risk categories of the non-polygenic models to values that are determined based on observed medical/polygenic events. As a result, the noted embodiments of the present invention provide efficient (e.g., linear) techniques for bridging the gap between medical/polygenic domains and non-polygenic models, which in turn enables the utilization of efficient and/or effective non-polygenic predictive models in relation to polygenic prediction. This in turn increases the computational efficiency and/or the operational reliability of performing health-related predictive data analysis. By increasing the computational efficiency and/or the operational reliability of performing health-related predictive data analysis, various embodiments of the present invention address substantial technical challenges related to computational efficiency and/or operational reliability of various existing health-related predictive data analysis and make important technical contributions to improving health-related predictive data analysis techniques.

Other exemplary innovative aspects of various embodiments of the present invention are as follows: (i) various embodiments of the present invention propose techniques that are configured to determine a genetic risk score that integrates genetic, behavioral, and other health information; (ii) various embodiments of the present invention propose techniques that use genetic credit risk scores as a quality control filter for Polygenic Risk Score (PRS) generation techniques; (iii) various embodiments of the present invention propose techniques that use genetic credit risk scores in combination with existing PRS generation approaches; and (iv) various embodiments of the present invention disclose repurposing of models used in existing credit risk scenarios for use in relation to genetic risk scenarios.

Some of the exemplary advantages of various embodiments of the present invention are as follows: enhanced accuracy of polygenic risk score prediction for application in clinical decision support systems; increased accuracy due to utilizing medication adherence data and other health determinants in addition to genetic input data; the ability to include additional genetic risk factors, such as copy number variations (CNVs), which are established to have causal risk in many diseases (especially cancer), but cannot be included in existing PRS calculations; and creation of a compound risk score that includes behavioral features, environmental features, phenotype features, genetic risk features, and complex genetic features in a manner that is configured to create enhanced and more applicable risk scores for clinical utility.

Various embodiments of the present invention repurpose well known financial credit risk models and modify them to determine the genetic risk of a phenotype being expressed. Methods for determining an individual's credit risk have been established for many years, are well-validated, and the accuracy and predictive power of such models are well-known. Various embodiments of the present invention propose a unique and non-obvious correlation between key elements of these credit score models and quantifying the potential for a detrimental health condition. That credit risk is therefore deemed to be an analogue of the risk of that detrimental health condition occurring (i.e. the borrower will default).

### II. DEFINITIONS

The term “initial risk scoring model” may refer to a data object that describes a model that is configured to process initial risk category values associated with a group of initial risk categories in order to generate a risk prediction, where the risk prediction is not a polygenic risk score prediction. Accordingly, the initial risk scoring model is associated with a predictive domain that is distinct from a polygenic risk scoring predictive domain. An example of an initial risk scoring model is a credit risk scoring model (such as a Fair, Isaac, and Company (FICO) credit risk coring model, a Black-Scholes credit risk scoring model, and/or the like) that is configured to process input values associated with a target individual's financial/credit history in order to generate a credit risk score for the target individual. In the noted example, the initial risk scoring model may be associated with a credit risk scoring predictive domain which is distinct from a polygenic risk scoring predictive domain. However, while various embodiments of the present invention are described with reference to initial risk scoring models that are credit risk scoring models, a person of ordinary skill in the art will recognize that other types of risk scoring models that are associated with predictive domains other than credit risk scoring predictive domains may be utilized in accordance with various embodiments of the present invention. In some embodiments, the initial risk scoring model is a logistic regression model.

The term “cross-domain mapping” may refer to a data object that describes mappings between the initial risk categories of a corresponding initial risk scoring model and inferred risk categories that are associated with a predictive domain that is distinct from the predictive domain of the corresponding initial risk scoring model. Accordingly, the cross-domain mapping describes mappings that enable using an initial risk scoring model in a predictive domain that is distinct from the primary predictive domain that is associated with the initial risk scoring model. For example, if the initial risk scoring model is a credit risk scoring model that is associated with a credit risk scoring predictive domain, the cross-domain mapping for the noted credit risk scoring model may map the credit risk scoring categories of the credit risk scoring model to inferred risk scoring categories that are derived from medical (e.g., polygenic data, other genetic variant data such as electronic medical record (EMR) data, and/or the like) record of target individuals. In the noted example, the noted cross-domain mapping enables using a credit risk scoring model for performing health-related predictive data analysis operations.

The term “compliance history category” may refer to a data object that describes an initial risk category for an initial risk scoring model that represents a property related to compliance of a target individual with one or more desired attributes during a particular historical timeframe (e.g., during the last ten years, for all of the period of availability of compliance history data, and/or the like), where the desired attributes are configured to be predicted by the initial risk scoring model. An example of a compliance history category is an initial risk category that describes a payment history of a particular target individual, such as a payment history category that describes the number of months since the month of the most recent financially derogatory record (e.g., the most recent debt nonpayment record) for the particular target individual. In some of the noted exemplary embodiments: (i) if the target individual is not associated with any derogatory records during the particular historical timeframe, the compliance history category is assigned a highest compliance history category value (e.g., a compliance history category value of 75); (ii) if the number of months since the month of the most recent financially derogatory record for the target individual is more than or equal to a first threshold number of months (e.g., 24 months), the compliance history category is assigned a second highest compliance history category value (e.g., a compliance history category value of 55); (iii) if the number of months since the month of the most recent financially derogatory record for the target individual is less than the first threshold number of months but more than or equal to a second threshold number of months (e.g., 12 months), the compliance history category is assigned a third highest compliance history category value (e.g., a compliance history category value of 25); (iv) if the number of months since the month of the most recent financially derogatory record for the target individual is less than the second threshold number of months but more than or equal to a third threshold number of months (e.g., 6 months), the compliance history category is assigned a fourth highest compliance history category value (e.g., a compliance history category value of 15); and (v) if the number of months since the month of the most recent financially derogatory record for the target individual is less than the fourth threshold number of months but more than or equal to a fifth threshold number of months (e.g., 0 months), the compliance history category is assigned a fifth highest compliance history category value (e.g., a compliance history category value of 10). In some embodiments, the noted payment history category can be mapped to a medical history category as part of generating a cross-domain mapping for the credit risk modeling with respect to a polygenic risk scoring predictive domain.

The term “medical history category” may refer to a data object that describes an inferred risk category that represents a property related to one or more health-related events for a target individual during a particular historical timeframe (e.g., during the last ten years, for all of the period of availability of medical history data, and/or the like). Examples of health-related events that can be captured by a medical history category may include: medical symptom history (e.g., data about severity of medical symptoms of the target individual over the particular historical timeframe), genetic variation data (e.g., data about single-nucleotide polymorphisms (SNPs) and/or CNVs that are present in the genome of the target individual), and/or the like. In some embodiments, a medical history category value for the medical history category may be determined based on at least one of the following: a trained generalized linear model (GLM) that is configured to process the medical symptom history data associated with the target individual in order to generate a medical symptom history representation for the target individual, and a non-linear predictive model that is configured to process the genetic variation data (e.g., the CNV data) associated with the target individual in order to generate a genetic variation representation for the target individual.

The term “record magnitude category” may refer to a data object that describes an initial risk category for an initial risk scoring model that represents a property related to a total value of records associated with a target individual during a current time. Examples of record magnitude categories include an initial risk category for a credit risk scoring model that describes a measure related to magnitude of outstanding debt of the target individual during the particular historical timeframe, such as a measure of the average balance of revolving trades of the target individual. In some of the noted exemplary embodiments: (i) if the average balance of revolving trade of the target individual is more than or equal to a first threshold (e.g., $1000), the record magnitude history category value for the record magnitude category of the target individual is assigned a lowest value (e.g., a value of 15); (ii) if the average balance of revolving trade of the target individual is less than the first threshold but more than or equal to a second threshold (e.g., $750), the record magnitude history category value for the record magnitude category of the target individual is assigned a second lowest value (e.g., a value of 25); (iii) if the average balance of revolving trade of the target individual is less than the second threshold but more than or equal to a third threshold (e.g., $500), the record magnitude history category value for the record magnitude category of the target individual is assigned a third lowest value (e.g., a value of 40); (iv) if the average balance of revolving trade of the target individual is less than the third threshold but more than or equal to a fourth threshold (e.g., $100), the record magnitude history category value for the record magnitude category of the target individual is assigned a fourth lowest value (e.g., a value of 50); (v) if the average balance of revolving trade of the target individual is less than the fourth threshold but more than or equal to a fourth threshold (e.g., $1), the record magnitude history category value for the record magnitude category of the target individual is assigned a fifth lowest value (e.g., a value of 65); (vi) if the average balance of revolving trade of the target individual is zero, the record magnitude history category value for the record magnitude category of the target individual is assigned a sixth value (e.g., a value of 55); and (vii) if the target individual has no revolving trades, the record magnitude history category value for the record magnitude category of the target individual is assigned a seventh value (e.g., a value of 30). In some embodiments, the noted record magnitude category can be mapped to a current phenotype category as part of generating a cross-domain mapping for the credit risk modeling with respect to a polygenic risk scoring predictive domain.

The term “current phenotype category” may refer to a data object that describes an inferred risk category that relates to current phenotypes (e.g., current diagnoses, current observed medical conditions, current observed behaviors, current observed appearance features, and/or the like) of a target individual during a current time. In some of the noted embodiments, a current phenotype category provides a measure of current genomic utilization of a target individual that can in turn be mapped to a measure of credit utilization of the target individual (e.g., an outstanding debt measure of the target individual). In some embodiments, the current phenotype category value for the current phenotype category is determined using a GLM model. In some embodiments, the current phenotype category value for the current phenotype category is determined using a non-linear predictive model, such as a Bell curve regression model.

The term “record history length category” may refer to a data object that describes an initial risk category for an initial risk scoring model that represents a property related to a total length of available and eligible input data for a target individual in order to generate initial risk predictions by the initial risk scoring model. For example, if the initial risk scoring model is a credit risk scoring model that is configured to generate credit risk predictions using all available credit history data within a defined historical timeframe (e.g., within the last ten years), the record history length category value for the record history length category of the target individual may be determined based on a measure of length of the available credit history of the target individual within the last years. In some of the noted exemplary embodiments: (i) if the measure of length of the available credit history of the target individual falls below a first threshold (e.g., 12 months), the record history length category value for record history length category of the target individual may be assigned a lowest value (e.g., a value of 12); (ii) if the measure of length of the available credit history of the target individual falls more than or equal to the first threshold but less than a second threshold (e.g., 24 months), the record history length category value for record history length category of the target individual may be assigned a second lowest value (e.g., a value of 35); (iii) if the measure of length of the available credit history of the target individual falls more than or equal to the second threshold but less than a third threshold (e.g., 47 months), the record history length category value for record history length category of the target individual may be assigned a third lowest value (e.g., a value of 60); and (iv) if the measure of length of the available credit history of the target individual falls more than or equal to the third threshold, the record history length category value for record history length category of the target individual may be assigned a fourth lowest value (e.g., a value of 75). In some embodiments, the noted record length history category can be mapped to a target condition onset delay category as part of generating a cross-domain mapping for the credit risk modeling with respect to a polygenic risk scoring predictive domain.

The term “target condition onset delay category” may refer to a data object that describes an inferred risk category that relates to a magnitude of the temporal interval between an estimated onset point in time for a corresponding target condition in a target individual and a current individual. The target condition onset delay category value for the target condition onset delay category may be determined based on a length of time related to management of the corresponding target condition (e.g., a corresponding disease, a corresponding phenotype, and/or the like). In some embodiments, the target condition onset delay category value for the target condition onset delay category may be determined using a GLM that is configured to generate positive values.

The term “record diversity category” may refer to a data object that describes an initial risk category for an initial risk scoring model that represents a property related to a number of record sources associated an activity record utilized by the initial risk scoring model to generate initial risk predictions. For example, if the initial risk scoring model is a credit risk scoring model, the record diversity category value for the record diversity category may describe a number of bankcard trade lines associated with a corresponding credit history during a current time and/or during a particular historical timeframe. In some of the noted exemplary embodiments: (i) if the number of bankcard trade lines is less than a first threshold (e.g., one), the record diversity category value for the record diversity category may be assigned a lowest value (e.g., a value of 15); (ii) if the number of bankcard trade lines is more than or equal to the first threshold but less than a second threshold (e.g., two), the record diversity category value for the record diversity category may be assigned a second lowest value (e.g., a value of 25); (iii) if the number of bankcard trade lines is more than or equal to the second threshold but less than or equal to a third threshold (e.g., three), the record diversity category value for the record diversity category may be assigned a third lowest value (e.g., a value of 50); (iv) if the number of bankcard trade lines is more than or equal to the third threshold but less than a fourth threshold (e.g., four), the record diversity category value for the record diversity category may be assigned a fourth lowest value (e.g., a value of 60); and (v) if the number of bankcard trade lines during the last six months is more than or equal to the fourth threshold, the record diversity category value for the record diversity category may be assigned a fifth lowest value (e.g., a value of 50). In some embodiments, the record diversity category can be mapped to a current therapeutic management category as part of generating a cross-domain mapping for the credit risk modeling with respect to a polygenic risk scoring predictive domain.

The term “current therapeutic management category” may refer to a data object that describes an inferred risk category that relates to a current therapeutic approach to a target condition of a target individual. For example, the current therapeutic management category may relate to a current disease management and/or a current medication adherence of a target individual with respect to a target condition. In some embodiments, the current therapeutic management category value for the current therapeutic management category is determined based on at least one of the following: (i) the polychronic diseases present in the target individual and their associated comorbidity in relation to the target condition, (ii) a measure of wellness/lifestyle of the target individual, and (iii) a measure of adherence of the target individual to medical and/or pharmaceutical guidelines for prevention and/or treatment of the target condition. In some embodiments, the current therapeutic management category value for the current therapeutic management category is determined using a GLM. In some embodiments, at least a portion of the data used to determine the current therapeutic management category value for the current therapeutic management category is generated using a non-linear prediction model, such as non-linear RX adherence prediction machine learning model and/or an RX adherence prediction deep learning model.

The term “query frequency category” may refer to a data object that describes an initial risk category for an initial risk scoring model that represents a property related to a recency of obtaining an initial risk prediction by the initial risk scoring model and/or to frequency of obtaining an initial risk prediction by the initial risk scoring model within a particular historical timeframe (e.g., within the last six months). For example, if the initial risk scoring model is a credit risk scoring model, the query frequency category value for the query frequency category may describe the number of credit inquiries performed using the credit risk scoring model during the last six months. In some of the noted exemplary embodiments: (i) if the number of the new credit inquiries during the last six months is less than a first threshold (e.g., one), the query frequency category value for the query frequency category may be assigned a highest value (e.g., a value of 70); (ii) if the number of the new credit inquiries during the last six months is more than or equal to the first threshold but less than a second threshold (e.g., two), the query frequency category value for the query frequency category may be assigned a second highest value (e.g., a value of 60); (iii) if the number of the new credit inquiries during the last six months is more than or equal to the second threshold but less than or equal to a third threshold (e.g., three), the query frequency category value for the query frequency category may be assigned a third highest value (e.g., a value of 45); (iv) if the number of the new credit inquiries during the last six months is more than or equal to the third threshold but less than a fourth threshold (e.g., four), the query frequency category value for the query frequency category may be assigned a fourth highest value (e.g., a value of 25); and (v) if the number of the new credit inquiries during the last six months is more than or equal to the fourth threshold, the query frequency category value for the query frequency category may be assigned a fifth highest value (e.g., a value of 20). In some embodiments, the query frequency category can be mapped to a genetic variance category as part of generating a cross-domain mapping for the credit risk modeling with respect to a polygenic risk scoring predictive domain.

The term “genetic variance category” may refer to a data object that describes an inferred risk category that relates to a variation of at least a portion of a genetic composition of a target individual relative to genetic population of an observed population and/or relative a current human genome reference. In some of the noted embodiments, the genetic variance category value for the genetic variance category is determined based on at least one of: (i) the number of genetic and/or medical tests performed during a historical timeframe, (ii) the identity of panels screened during the noted genetic and/or medical texts, and (iii) any VUSs found during the noted genetic and/or medical texts. In some embodiments, the genetic variance category value for the genetic variance category is determined using a GLM. In some embodiments, the genetic variance category value for the genetic variance category is determined using a non-linear prediction model. In some embodiments, the genetic variance category value for the genetic variance category is determined using a VUS probability distribution, such as a VUS probability that relates clinical significance of particular VUSs with respect to particular target conditions.

The term “inferred risk category value” may refer to a data object that describes a singular value and/or a singular vector that contains information related to a corresponding inferred risk category configured to be transferred as inputs to an initial risk scoring model. Accordingly, the inferred risk category value is a mapping of selected information from a secondary predictive domain other than the default predictive domain of the initial risk scoring model (e.g., from the polygenic risk scoring predictive domain, which may be distinct from the predictive domain of an initial risk scoring model) to a variable of the initial risk scoring model. For example, given a medical history category as an inferred risk category, the inferred risk category value for the noted medical history category may describe the sets of medical history events that are encoded into a common representation (e.g., into a common scalar representation) in order to input to an initial risk scoring (e.g., to a credit risk scoring model).

The term “per-category weight value” may refer to a data object that describes an estimated significance of a corresponding inferred risk category value for a corresponding inferred risk category to determining a health-related risk prediction for a target individual with respect to a target condition. In some of the noted embodiments, the per-category weight values provide a technique through which developers of health-related predictive data analysis models can transfer domain-level information about relationships between observed variables and target conditions to domain-agnostic and/or domain-alien initial risk scoring models, such as credit risk scoring models in relation to health-related predictive data analysis models. For example, the medical history category value for the medical history category may be deemed more pertinent for a first target condition (e.g., diabetes) relative to a second target condition (e.g., acquired immunodeficiency syndrome (AIDS)). In the noted example, the per-category weight value for the medical history category relative to the first target condition will likely be higher than the per-category weight value for the medical history category relative the second target condition. As another example, the genetic variation category value for the genetic variation category may be deemed more pertinent for a first target condition (e.g., hemophilia) relative to a second target condition (e.g., common cold). In the noted example, the per-category weight value for the genetic variation category relative to the first target condition will likely be higher than the per-category weight value for the genetic variation category relative the second target condition.

### III. COMPUTER PROGRAM PRODUCTS, METHODS, AND COMPUTING ENTITIES

Embodiments of the present invention may be implemented in various ways, including as computer program products that comprise articles of manufacture. Such computer program products may include one or more software components including, for example, software objects, methods, data structures, or the like. A software component may be coded in any of a variety of programming languages. An illustrative programming language may be a lower-level programming language such as an assembly language associated with a particular hardware architecture and/or operating system platform. A software component comprising assembly language instructions may require conversion into executable machine code by an assembler prior to execution by the hardware architecture and/or platform. Another example programming language may be a higher-level programming language that may be portable across multiple architectures. A software component comprising higher-level programming language instructions may require conversion to an intermediate representation by an interpreter or a compiler prior to execution.

Other examples of programming languages include, but are not limited to, a macro language, a shell or command language, a job control language, a script language, a database query or search language, and/or a report writing language. In one or more example embodiments, a software component comprising instructions in one of the foregoing examples of programming languages may be executed directly by an operating system or other software component without having to be first transformed into another form. A software component may be stored as a file or other data storage construct. Software components of a similar type or functionally related may be stored together such as, for example, in a particular directory, folder, or library. Software components may be static (e.g., pre-established or fixed) or dynamic (e.g., created or modified at the time of execution).

A computer program product may include a non-transitory computer-readable storage medium storing applications, programs, program modules, scripts, source code, program code, object code, byte code, compiled code, interpreted code, machine code, executable instructions, and/or the like (also referred to herein as executable instructions, instructions for execution, computer program products, program code, and/or similar terms used herein interchangeably). Such non-transitory computer-readable storage media include all computer-readable media (including volatile and non-volatile media).

In one embodiment, a non-volatile computer-readable storage medium may include a floppy disk, flexible disk, hard disk, solid-state storage (SSS) (e.g., a solid state drive (SSD), solid state card (SSC), solid state module (SSM), enterprise flash drive, magnetic tape, or any other non-transitory magnetic medium, and/or the like. A non-volatile computer-readable storage medium may also include a punch card, paper tape, optical mark sheet (or any other physical medium with patterns of holes or other optically recognizable indicia), compact disc read only memory (CD-ROM), compact disc-rewritable (CD-RW), digital versatile disc (DVD), Blu-ray disc (BD), any other non-transitory optical medium, and/or the like. Such a non-volatile computer-readable storage medium may also include read-only memory (ROM), programmable read-only memory (PROM), erasable programmable read-only memory (EPROM), electrically erasable programmable read-only memory (EEPROM), flash memory (e.g., Serial, NAND, NOR, and/or the like), multimedia memory cards (MMC), secure digital (SD) memory cards, SmartMedia cards, CompactFlash (CF) cards, Memory Sticks, and/or the like. Further, a non-volatile computer-readable storage medium may also include conductive-bridging random access memory (CBRAM), phase-change random access memory (PRAM), ferroelectric random-access memory (FeRAM), non-volatile random-access memory (NVRAM), magnetoresistive random-access memory (MRAM), resistive random-access memory (RRAM), Silicon-Oxide-Nitride-Oxide-Silicon memory (SONOS), floating junction gate random access memory (FJG RAM), Millipede memory, racetrack memory, and/or the like.

In one embodiment, a volatile computer-readable storage medium may include random access memory (RAM), dynamic random access memory (DRAM), static random access memory (SRAM), fast page mode dynamic random access memory (FPM DRAM), extended data-out dynamic random access memory (EDO DRAM), synchronous dynamic random access memory (SDRAM), double data rate synchronous dynamic random access memory (DDR SDRAM), double data rate type two synchronous dynamic random access memory (DDR2 SDRAM), double data rate type three synchronous dynamic random access memory (DDR3 SDRAM), Rambus dynamic random access memory (RDRAM), Twin Transistor RAM (TTRAM), Thyristor RAM (T-RAM), Zero-capacitor (Z-RAM), Rambus in-line memory module (RIMM), dual in-line memory module (DIMM), single in-line memory module (SIMM), video random access memory (VRAM), cache memory (including various levels), flash memory, register memory, and/or the like. It will be appreciated that where embodiments are described to use a computer-readable storage medium, other types of computer-readable storage media may be substituted for or used in addition to the computer-readable storage media described above.

As should be appreciated, various embodiments of the present invention may also be implemented as methods, apparatus, systems, computing devices, computing entities, and/or the like. As such, embodiments of the present invention may take the form of an apparatus, system, computing device, computing entity, and/or the like executing instructions stored on a computer-readable storage medium to perform certain steps or operations. Thus, embodiments of the present invention may also take the form of an entirely hardware embodiment, an entirely computer program product embodiment, and/or an embodiment that comprises combination of computer program products and hardware performing certain steps or operations. Embodiments of the present invention are described below with reference to block diagrams and flowchart illustrations. Thus, it should be understood that each block of the block diagrams and flowchart illustrations may be implemented in the form of a computer program product, an entirely hardware embodiment, a combination of hardware and computer program products, and/or apparatus, systems, computing devices, computing entities, and/or the like carrying out instructions, operations, steps, and similar words used interchangeably (e.g., the executable instructions, instructions for execution, program code, and/or the like) on a computer-readable storage medium for execution. For example, retrieval, loading, and execution of code may be performed sequentially such that one instruction is retrieved, loaded, and executed at a time. In some exemplary embodiments, retrieval, loading, and/or execution may be performed in parallel such that multiple instructions are retrieved, loaded, and/or executed together. Thus, such embodiments can produce specifically-configured machines performing the steps or operations specified in the block diagrams and flowchart illustrations. Accordingly, the block diagrams and flowchart illustrations support various combinations of embodiments for performing the specified instructions, operations, or steps.

### IV. EXEMPLARY SYSTEM ARCHITECTURE

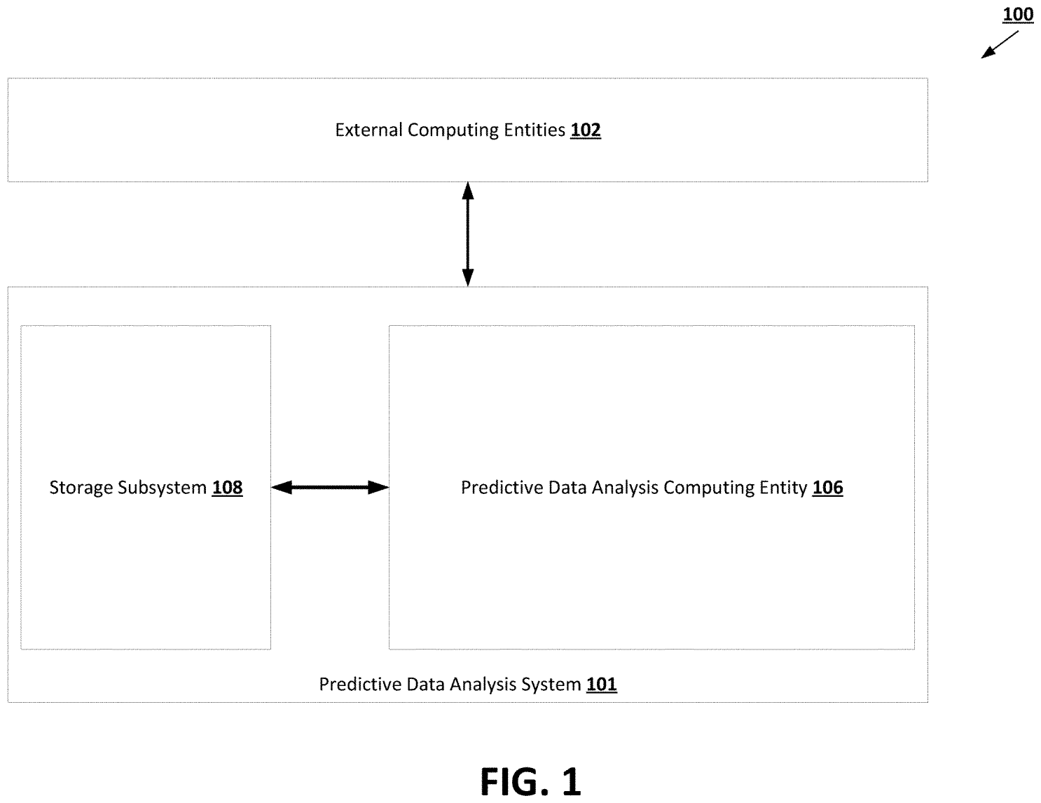

FIG. 1 is a schematic diagram of an example architecture 100 for performing health-related predictive data analysis. The architecture 100 includes a predictive data analysis system 101 configured to receive health-related predictive data analysis requests from external computing entities 102, process the predictive data analysis requests to generate health-related risk predictions, provide the generated health-related risk predictions to the external computing entities 102, and automatically perform prediction-based actions based at least in part on the generated polygenic risk score predictions. Examples of health-related predictions include genetic risk predictions, polygenic risk predictions, medical risk predictions, clinical risk predictions, behavioral risk predictions, and/or the like.

In some embodiments, predictive data analysis system 101 may communicate with at least one of the external computing entities 102 using one or more communication networks. Examples of communication networks include any wired or wireless communication network including, for example, a wired or wireless local area network (LAN), personal area network (PAN), metropolitan area network (MAN), wide area network (WAN), or the like, as well as any hardware, software and/or firmware required to implement it (such as, e.g., network routers, and/or the like).

The predictive data analysis system 101 may include a predictive data analysis computing entity 106 and a storage subsystem 108. The predictive data analysis computing entity 106 may be configured to receive health-related predictive data analysis requests from one or more external computing entities 102, process the predictive data analysis requests to generate the polygenic risk score predictions corresponding to the predictive data analysis requests, provide the generated polygenic risk score predictions to the external computing entities 102, and automatically perform prediction-based actions based at least in part on the generated polygenic risk score predictions.

The storage subsystem 108 may be configured to store input data used by the predictive data analysis computing entity 106 to perform health-related predictive data analysis as well as model definition data used by the predictive data analysis computing entity 106 to perform various health-related predictive data analysis tasks. The storage subsystem 108 may include one or more storage units, such as multiple distributed storage units that are connected through a computer network. Each storage unit in the storage subsystem 108 may store at least one of one or more data assets and/or one or more data about the computed properties of one or more data assets. Moreover, each storage unit in the storage subsystem 108 may include one or more non-volatile storage or memory media including but not limited to hard disks, ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like.

### Exemplary Predictive Data Analysis Computing Entity

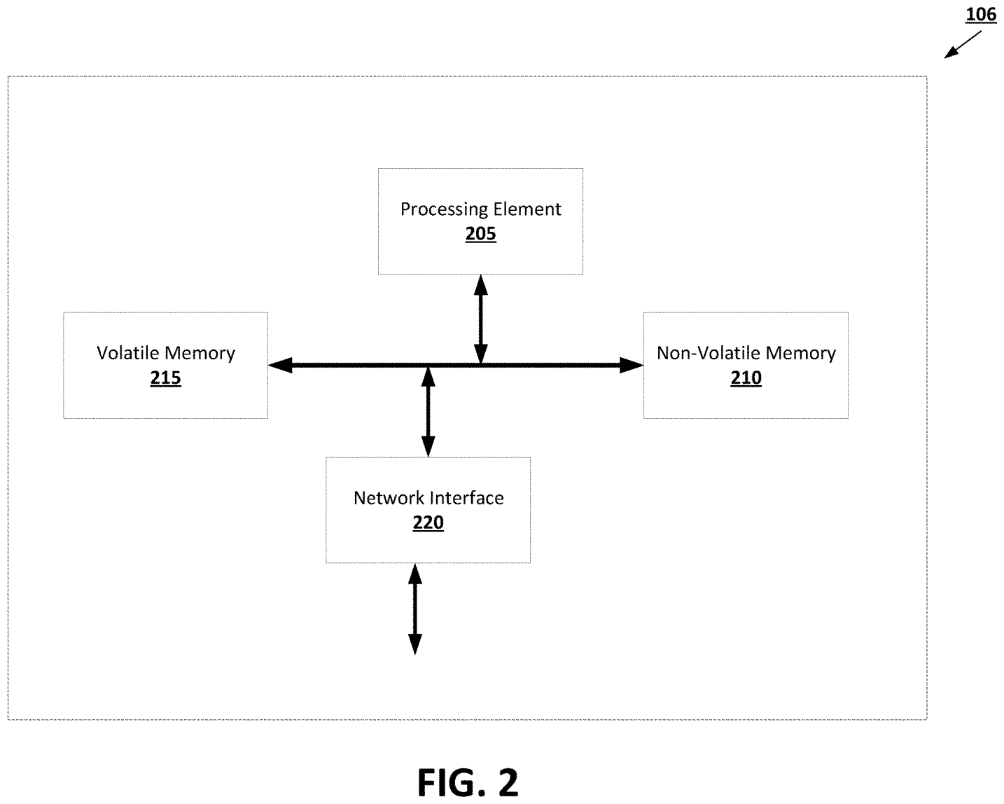

FIG. 2 provides a schematic of a predictive data analysis computing entity 106 according to one embodiment of the present invention. In general, the terms computing entity, computer, entity, device, system, and/or similar words used herein interchangeably may refer to, for example, one or more computers, computing entities, desktops, mobile phones, tablets, phablets, notebooks, laptops, distributed systems, kiosks, input terminals, servers or server networks, blades, gateways, switches, processing devices, processing entities, set-top boxes, relays, routers, network access points, base stations, the like, and/or any combination of devices or entities adapted to perform the functions, operations, and/or processes described herein. Such functions, operations, and/or processes may include, for example, transmitting, receiving, operating on, processing, displaying, storing, determining, creating/generating, monitoring, evaluating, comparing, and/or similar terms used herein interchangeably. In one embodiment, these functions, operations, and/or processes can be performed on data, content, information, and/or similar terms used herein interchangeably.

As indicated, in one embodiment, the predictive data analysis computing entity 106 may also include one or more communications interfaces 220 for communicating with various computing entities, such as by communicating data, content, information, and/or similar terms used herein interchangeably that can be transmitted, received, operated on, processed, displayed, stored, and/or the like.

As shown in FIG. 2, in one embodiment, the predictive data analysis computing entity 106 may include or be in communication with one or more processing elements 205 (also referred to as processors, processing circuitry, and/or similar terms used herein interchangeably) that communicate with other elements within the predictive data analysis computing entity 106 via a bus, for example. As will be understood, the processing element 205 may be embodied in a number of different ways.

For example, the processing element 205 may be embodied as one or more complex programmable logic devices (CPLDs), microprocessors, multi-core processors, coprocessing entities, application-specific instruction-set processors (ASIPs), microcontrollers, and/or controllers. Further, the processing element 205 may be embodied as one or more other processing devices or circuitry. The term circuitry may refer to an entirely hardware embodiment or a combination of hardware and computer program products. Thus, the processing element 205 may be embodied as integrated circuits, application specific integrated circuits (ASICs), field programmable gate arrays (FPGAs), programmable logic arrays (PLAs), hardware accelerators, other circuitry, and/or the like.

As will therefore be understood, the processing element 205 may be configured for a particular use or configured to execute instructions stored in volatile or non-volatile media or otherwise accessible to the processing element 205. As such, whether configured by hardware or computer program products, or by a combination thereof, the processing element 205 may be capable of performing steps or operations according to embodiments of the present invention when configured accordingly.

In one embodiment, the predictive data analysis computing entity 106 may further include or be in communication with non-volatile media (also referred to as non-volatile storage, memory, memory storage, memory circuitry and/or similar terms used herein interchangeably). In one embodiment, the non-volatile storage or memory may include one or more non-volatile storage or memory media 210, including but not limited to hard disks, ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like.

As will be recognized, the non-volatile storage or memory media may store databases, database instances, database management systems, data, applications, programs, program modules, scripts, source code, object code, byte code, compiled code, interpreted code, machine code, executable instructions, and/or the like. The term database, database instance, database management system, and/or similar terms used herein interchangeably may refer to a collection of records or data that is stored in a computer-readable storage medium using one or more database models, such as a hierarchical database model, network model, relational model, entity-relationship model, object model, document model, semantic model, graph model, and/or the like.

In one embodiment, the predictive data analysis computing entity 106 may further include or be in communication with volatile media (also referred to as volatile storage, memory, memory storage, memory circuitry and/or similar terms used herein interchangeably). In one embodiment, the volatile storage or memory may also include one or more volatile storage or memory media 215, including but not limited to RAM, DRAM, SRAM, FPM DRAM, EDO DRAM, SDRAM, DDR SDRAM, DDR2 SDRAM, DDR3 SDRAM, RDRAM, TTRAM, T-RAM, Z-RAM, RIMM, DIMM, SIMM, VRAM, cache memory, register memory, and/or the like.

As will be recognized, the volatile storage or memory media may be used to store at least portions of the databases, database instances, database management systems, data, applications, programs, program modules, scripts, source code, object code, byte code, compiled code, interpreted code, machine code, executable instructions, and/or the like being executed by, for example, the processing element 205. Thus, the databases, database instances, database management systems, data, applications, programs, program modules, scripts, source code, object code, byte code, compiled code, interpreted code, machine code, executable instructions, and/or the like may be used to control certain aspects of the operation of the predictive data analysis computing entity 106 with the assistance of the processing element 205 and operating system.

As indicated, in one embodiment, the predictive data analysis computing entity 106 may also include one or more communications interfaces 220 for communicating with various computing entities, such as by communicating data, content, information, and/or similar terms used herein interchangeably that can be transmitted, received, operated on, processed, displayed, stored, and/or the like. Such communication may be executed using a wired data transmission protocol, such as fiber distributed data interface (FDDI), digital subscriber line (DSL), Ethernet, asynchronous transfer mode (ATM), frame relay, data over cable service interface specification (DOCSIS), or any other wired transmission protocol. Similarly, the predictive data analysis computing entity 106 may be configured to communicate via wireless external communication networks using any of a variety of protocols, such as general packet radio service (GPRS), Universal Mobile Telecommunications System (UMTS), Code Division Multiple Access 2000 (CDMA2000), CDMA2000 1× (1×RTT), Wideband Code Division Multiple Access (WCDMA), Global System for Mobile Communications (GSM), Enhanced Data rates for GSM Evolution (EDGE), Time Division-Synchronous Code Division Multiple Access (TD-SCDMA), Long Term Evolution (LTE), Evolved Universal Terrestrial Radio Access Network (E-UTRAN), Evolution-Data Optimized (EVDO), High Speed Packet Access (HSPA), High-Speed Downlink Packet Access (HSDPA), IEEE 802.11 (Wi-Fi), Wi-Fi Direct, 802.16 (WiMAX), ultra-wideband (UWB), infrared (IR) protocols, near field communication (NFC) protocols, Wibree, Bluetooth protocols, wireless universal serial bus (USB) protocols, and/or any other wireless protocol.

Although not shown, the predictive data analysis computing entity 106 may include or be in communication with one or more input elements, such as a keyboard input, a mouse input, a touch screen/display input, motion input, movement input, audio input, pointing device input, joystick input, keypad input, and/or the like. The predictive data analysis computing entity 106 may also include or be in communication with one or more output elements (not shown), such as audio output, video output, screen/display output, motion output, movement output, and/or the like.

### Exemplary External Computing Entity

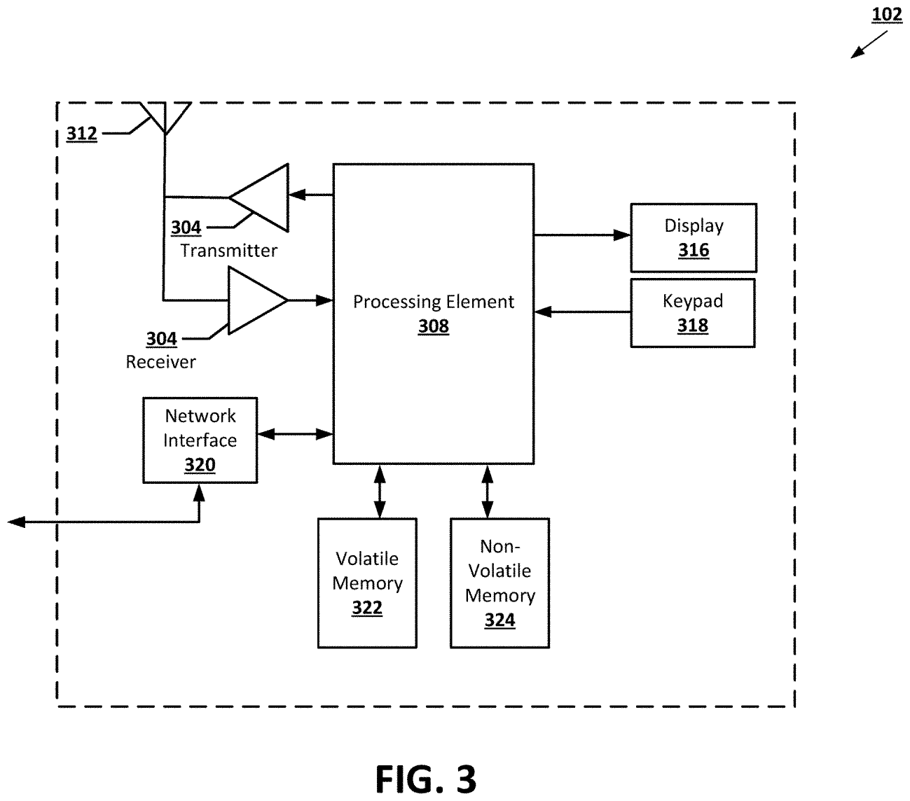

FIG. 3 provides an illustrative schematic representative of an external computing entity 102 that can be used in conjunction with embodiments of the present invention. In general, the terms device, system, computing entity, entity, and/or similar words used herein interchangeably may refer to, for example, one or more computers, computing entities, desktops, mobile phones, tablets, phablets, notebooks, laptops, distributed systems, kiosks, input terminals, servers or server networks, blades, gateways, switches, processing devices, processing entities, set-top boxes, relays, routers, network access points, base stations, the like, and/or any combination of devices or entities adapted to perform the functions, operations, and/or processes described herein. External computing entities 102 can be operated by various parties. As shown in FIG. 3, the external computing entity 102 can include an antenna 312, a transmitter 304 (e.g., radio), a receiver 306 (e.g., radio), and a processing element 308 (e.g., CPLDs, microprocessors, multi-core processors, coprocessing entities, ASIPs, microcontrollers, and/or controllers) that provides signals to and receives signals from the transmitter 304 and receiver 306, correspondingly.

The signals provided to and received from the transmitter 304 and the receiver 306, correspondingly, may include signaling information/data in accordance with air interface standards of applicable wireless systems. In this regard, the external computing entity 102 may be capable of operating with one or more air interface standards, communication protocols, modulation types, and access types. More particularly, the external computing entity 102 may operate in accordance with any of a number of wireless communication standards and protocols, such as those described above with regard to the predictive data analysis computing entity 106. In a particular embodiment, the external computing entity 102 may operate in accordance with multiple wireless communication standards and protocols, such as UMTS, CDMA2000, 1×RTT, WCDMA, GSM, EDGE, TD-SCDMA, LTE, E-UTRAN, EVDO, HSPA, HSDPA, Wi-Fi, Wi-Fi Direct, WiMAX, UWB, IR, NFC, Bluetooth, USB, and/or the like. Similarly, the external computing entity 102 may operate in accordance with multiple wired communication standards and protocols, such as those described above with regard to the predictive data analysis computing entity 106 via a network interface 320.

Via these communication standards and protocols, the external computing entity 102 can communicate with various other entities using concepts such as Unstructured Supplementary Service Data (USSD), Short Message Service (SMS), Multimedia Messaging Service (MMS), Dual-Tone Multi-Frequency Signaling (DTMF), and/or Subscriber Identity Module Dialer (SIM dialer). The external computing entity 102 can also download changes, add-ons, and updates, for instance, to its firmware, software (e.g., including executable instructions, applications, program modules), and operating system.

According to one embodiment, the external computing entity 102 may include location determining aspects, devices, modules, functionalities, and/or similar words used herein interchangeably. For example, the external computing entity 102 may include outdoor positioning aspects, such as a location module adapted to acquire, for example, latitude, longitude, altitude, geocode, course, direction, heading, speed, universal time (UTC), date, and/or various other information/data. In one embodiment, the location module can acquire data, sometimes known as ephemeris data, by identifying the number of satellites in view and the relative positions of those satellites (e.g., using global positioning systems (GPS)). The satellites may be a variety of different satellites, including Low Earth Orbit (LEO) satellite systems, Department of Defense (DOD) satellite systems, the European Union Galileo positioning systems, the Chinese Compass navigation systems, Indian Regional Navigational satellite systems, and/or the like. This data can be collected using a variety of coordinate systems, such as the Decimal Degrees (DD); Degrees, Minutes, Seconds (DMS); Universal Transverse Mercator (UTM); Universal Polar Stereographic (UPS) coordinate systems; and/or the like. Alternatively, the location information/data can be determined by triangulating the external computing entity's 102 position in connection with a variety of other systems, including cellular towers, Wi-Fi access points, and/or the like. Similarly, the external computing entity 102 may include indoor positioning aspects, such as a location module adapted to acquire, for example, latitude, longitude, altitude, geocode, course, direction, heading, speed, time, date, and/or various other information/data. Some of the indoor systems may use various position or location technologies including RFID tags, indoor beacons or transmitters, Wi-Fi access points, cellular towers, nearby computing devices (e.g., smartphones, laptops) and/or the like. For instance, such technologies may include the iBeacons, Gimbal proximity beacons, Bluetooth Low Energy (BLE) transmitters, NFC transmitters, and/or the like. These indoor positioning aspects can be used in a variety of settings to determine the location of someone or something to within inches or centimeters.

The external computing entity 102 may also comprise a user interface (that can include a display 316 coupled to a processing element 308) and/or a user input interface (coupled to a processing element 308). For example, the user interface may be a user application, browser, user interface, and/or similar words used herein interchangeably executing on and/or accessible via the external computing entity 102 to interact with and/or cause display of information/data from the predictive data analysis computing entity 106, as described herein. The user input interface can comprise any of a number of devices or interfaces allowing the external computing entity 102 to receive data, such as a keypad 318 (hard or soft), a touch display, voice/speech or motion interfaces, or other input device. In embodiments including a keypad 318, the keypad 318 can include (or cause display of) the conventional numeric (0-9) and related keys (#, *), and other keys used for operating the external computing entity 102 and may include a full set of alphabetic keys or set of keys that may be activated to provide a full set of alphanumeric keys. In addition to providing input, the user input interface can be used, for example, to activate or deactivate certain functions, such as screen savers and/or sleep modes.

The external computing entity 102 can also include volatile storage or memory 322 and/or non-volatile storage or memory 324, which can be embedded and/or may be removable. For example, the non-volatile memory may be ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like. The volatile memory may be RAM, DRAM, SRAM, FPM DRAM, EDO DRAM, SDRAM, DDR SDRAM, DDR2 SDRAM, DDR3 SDRAM, RDRAM, TTRAM, T-RAM, Z-RAM, RIMM, DIMM, SIMM, VRAM, cache memory, register memory, and/or the like. The volatile and non-volatile storage or memory can store databases, database instances, database management systems, data, applications, programs, program modules, scripts, source code, object code, byte code, compiled code, interpreted code, machine code, executable instructions, and/or the like to implement the functions of the external computing entity 102. As indicated, this may include a user application that is resident on the entity or accessible through a browser or other user interface for communicating with the predictive data analysis computing entity 106 and/or various other computing entities.

In another embodiment, the external computing entity 102 may include one or more components or functionality that are the same or similar to those of the predictive data analysis computing entity 106, as described in greater detail above. As will be recognized, these architectures and descriptions are provided for exemplary purposes only and are not limiting to the various embodiments.

In various embodiments, the external computing entity 102 may be embodied as an artificial intelligence (AI) computing entity, such as an Amazon Echo, Amazon Echo Dot, Amazon Show, Google Home, and/or the like. Accordingly, the external computing entity 102 may be configured to provide and/or receive information/data from a user via an input/output mechanism, such as a display, a camera, a speaker, a voice-activated input, and/or the like. In certain embodiments, an AI computing entity may comprise one or more predefined and executable program algorithms stored within an onboard memory storage module, and/or accessible over a network. In various embodiments, the AI computing entity may be configured to retrieve and/or execute one or more of the predefined program algorithms upon the occurrence of a predefined trigger event.

### V. EXEMPLARY SYSTEM OPERATIONS

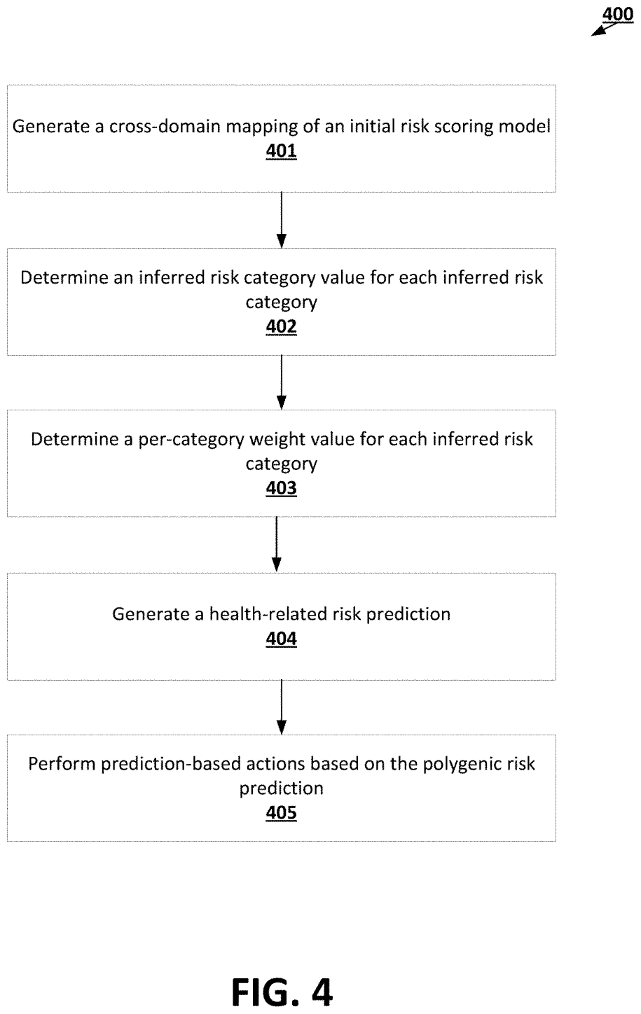

FIG. 4 is a flowchart diagram of an example process 400 for performing health-related predictive data analysis for a target individual with respect to a target condition (e.g., a target medical condition, such as a target disease). Via the various steps/operations of the process 400, the predictive data analysis computing entity 106 can perform cross-domain mapping to utilize more efficient and/or more reliable non-polygenic models in order to perform health-related predictive data analysis, which in turn increases the efficiency and/or reliability of performing the noted health-related predictive data analysis operations.

The process 400 begins at step/operation 401 when the predictive data analysis computing entity 106 generates a cross-domain mapping of an initial risk scoring model. In some embodiments, to generate the cross-domain mapping, the predictive data analysis computing entity 106 maps each risk category of the initial risk scoring model (i.e., each “initial risk category”) to an inferred risk category, where each inferred risk category is associated with one or more observed input variables of the target individual. Aspects of initial risk scoring models and cross-domain mappings are described in greater detail below.

In some embodiments, an initial risk scoring model describes a model that is configured to process initial risk category values associated with a group of initial risk categories in order to generate a risk prediction, where the risk prediction is not a polygenic risk score prediction. Accordingly, the initial risk scoring model is associated with a predictive domain that is distinct from a polygenic risk scoring predictive domain. An example of an initial risk scoring model is a credit risk scoring model (such as a FICO credit risk coring models, other models representing quantitative financial credit risk scenarios, and/or the like) that is configured to process input values associated with a target individual's financial/credit history in order to generate a credit risk score for the target individual. In the noted example, the initial risk scoring model may be associated with a credit risk scoring predictive domain which is distinct from a polygenic risk scoring predictive domain and/or a clinical risk scoring domain. However, while various embodiments of the present invention are described with reference to initial risk scoring models that are credit risk scoring models, a person of ordinary skill in the art will recognize that other types of risk scoring models that are associated with predictive domains other than credit risk scoring predictive domains may be utilized in accordance with various embodiments of the present invention. In some embodiments, the initial risk scoring model is a logistic regression model.

In some embodiments, a cross-domain mapping describes mappings between the initial risk categories of a corresponding initial risk scoring model and inferred risk categories that are associated with a predictive domain that is distinct from the predictive domain of the corresponding initial risk scoring model. Accordingly, the cross-domain mapping describes mappings that enable using an initial risk scoring model in a predictive domain that is distinct from the primary predictive domain that is associated with the initial risk scoring model. For example, if the initial risk scoring model is a credit risk scoring model that is associated with a credit risk scoring predictive domain, the cross-domain mapping for the noted credit risk scoring model may map the credit risk scoring categories of the credit risk scoring model to inferred risk scoring categories that are derived from medical (e.g., polygenic) record of target individuals. In the noted example, the noted cross-domain mapping enables using a credit risk scoring model for performing health-related predictive data analysis operations.

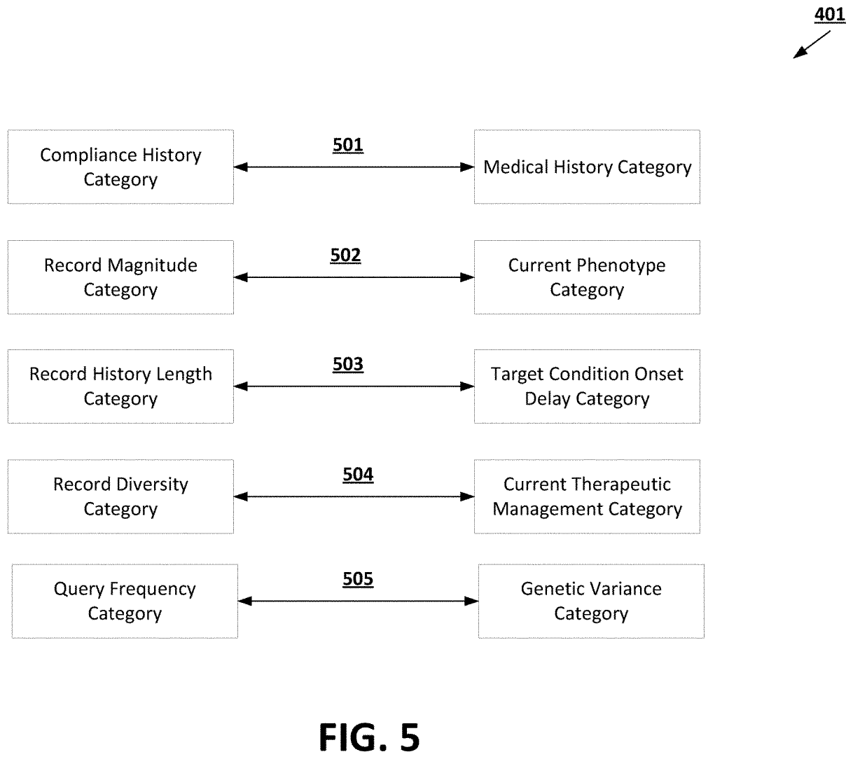

In some embodiments, step/operation 401 can be performed in accordance with the process that is depicted in FIG. 5. The process depicted in FIG. 5 begins at step/operation 501 when the predictive data analysis computing entity 106 maps a compliance history category of the initial risk scoring model to a medical history category. For example, the predictive data analysis computing entity 106 may map a payment history category associated with a credit risk scoring model to a medical history category. Aspects of compliance history categories and medical history categories are described in greater detail below.

In some embodiments, a compliance history category describes an initial risk category for an initial risk scoring model that represents a property related to compliance of a target individual with one or more desired attributes during a particular historical timeframe (e.g., during the last ten years, for all of the period of availability of compliance history data, and/or the like), where the desired attributes are configured to be predicted by the initial risk scoring model. An example of a compliance history category is an initial risk category that describes a payment history of a particular target individual, such as a payment history category that describes the number of months since the month of the most recent financially derogatory record (e.g., the most recent debt nonpayment record) for the particular target individual. In some of the noted exemplary embodiments: (i) if the target individual is not associated with any derogatory records during the particular historical timeframe, the compliance history category is assigned a highest compliance history category value (e.g., a compliance history category value of 75); (ii) if the number of months since the month of the most recent financially derogatory record for the target individual is more than or equal to a first threshold number of months (e.g., 24 months), the compliance history category is assigned a second highest compliance history category value (e.g., a compliance history category value of 55); (iii) if the number of months since the month of the most recent financially derogatory record for the target individual is less than the first threshold number of months but more than or equal to a second threshold number of months (e.g., 12 months), the compliance history category is assigned a third highest compliance history category value (e.g., a compliance history category value of 25); (iv) if the number of months since the month of the most recent financially derogatory record for the target individual is less than the second threshold number of months but more than or equal to a third threshold number of months (e.g., 6 months), the compliance history category is assigned a fourth highest compliance history category value (e.g., a compliance history category value of 15); and (v) if the number of months since the month of the most recent financially derogatory record for the target individual is less than the fourth threshold number of months but more than or equal to a fifth threshold number of months (e.g., 0 months), the compliance history category is assigned a fifth highest compliance history category value (e.g., a compliance history category value of 10). In some embodiments, the noted payment history category can be mapped to a medical history category as part of generating a cross-domain mapping for the credit risk modeling with respect to a polygenic risk scoring predictive domain.

In some embodiments, a medical history category describes an inferred risk category that represents a property related to one or more health-related events for a target individual during a particular historical timeframe (e.g., during the last ten years, for all of the period of availability of medical history data, and/or the like). Examples of health-related events that can be captured by a medical history category may include: medical symptom history (e.g., data about severity of medical symptoms of the target individual over the particular historical timeframe), genetic variation data (e.g., data about SNPs and/or CNVs that are present in the genome of the target individual, and/or the like. In some embodiments, a medical history category value for the medical history category may be determined based on at least one of the following: a trained GLM that is configured to process the medical symptom history data associated with the target individual in order to generate a medical symptom history representation for the target individual, and a non-linear predictive model that is configured to process the genetic variation data (e.g., the CNV data) associated with the target individual in order to generate a genetic variation representation for the target individual.

At step/operation 502, the predictive data analysis computing entity 106 maps a record magnitude category of the initial risk scoring model to a current phenotype category. For example, the predictive data analysis computing entity 106 may map an outstanding debt amount category associated with a credit risk scoring model to a current phenotype category. Aspects of record magnitude categories and current phenotype categories are described in greater detail below.

In some embodiments, a record magnitude category describes an initial risk category for an initial risk scoring model that represents a property related to a total value of records associated with a target individual during a current time. Examples of record magnitude categories include an initial risk category for a credit risk scoring model that describes a measure related to magnitude of outstanding debt of the target individual during the particular historical timeframe, such as a measure of the average balance of revolving trades of the target individual. In some of the noted exemplary embodiments: (i) if the average balance of revolving trade of the target individual is more than or equal to a first threshold (e.g., $1000), the record magnitude history category value for the record magnitude category of the target individual is assigned a lowest value (e.g., a value of 15); (ii) if the average balance of revolving trade of the target individual is less than the first threshold but more than or equal to a second threshold (e.g., $750), the record magnitude history category value for the record magnitude category of the target individual is assigned a second lowest value (e.g., a value of 25); (iii) if the average balance of revolving trade of the target individual is less than the second threshold but more than or equal to a third threshold (e.g., $500), the record magnitude history category value for the record magnitude category of the target individual is assigned a third lowest value (e.g., a value of 40); (iv) if the average balance of revolving trade of the target individual is less than the third threshold but more than or equal to a fourth threshold (e.g., $100), the record magnitude history category value for the record magnitude category of the target individual is assigned a fourth lowest value (e.g., a value of 50); (v) if the average balance of revolving trade of the target individual is less than the fourth threshold but more than or equal to a fourth threshold (e.g., $1), the record magnitude history category value for the record magnitude category of the target individual is assigned a fifth lowest value (e.g., a value of 65); (vi) if the average balance of revolving trade of the target individual is zero, the record magnitude history category value for the record magnitude category of the target individual is assigned a sixth value (e.g., a value of 55); and (vii) if the target individual has no revolving trades, the record magnitude history category value for the record magnitude category of the target individual is assigned a seventh value (e.g., a value of 30). In some embodiments, the noted record magnitude category can be mapped to a current phenotype category as part of generating a cross-domain mapping for the credit risk modeling with respect to a polygenic risk scoring predictive domain.

In some embodiments, a current phenotype category describes an inferred risk category that relates to current phenotypes (e.g., current diagnoses, current observed medical conditions, current observed behaviors, current observed appearance features, and/or the like) of a target individual during a current time. In some of the noted embodiments, a current phenotype category provides a measure of current genomic utilization of a target individual that can in turn be mapped to a measure of credit utilization of the target individual (e.g., an outstanding debt measure of the target individual). In some embodiments, the current phenotype category value for the current phenotype category is determined using a GLM model. In some embodiments, the current phenotype category value for the current phenotype category is determined using a non-linear predictive model, such as a Bell curve regression model.

At step/operation 503, the predictive data analysis computing entity 106 maps a record history length category of the initial risk scoring model to a target condition onset delay category. For example, the predictive data analysis computing entity 106 may map a credit report history length category associated with a credit risk scoring model to a target condition onset delay category. Aspects of record history length categories and target condition onset delay categories are described in greater detail below.

In some embodiments, a record history length category describes an initial risk category for an initial risk scoring model that represents a property related to a total length of available and eligible input data for a target individual in order to generate initial risk predictions by the initial risk scoring model. For example, if the initial risk scoring model is a credit risk scoring model that is configured to generate credit risk predictions using all available credit history data within a defined historical timeframe (e.g., within the last ten years), the record history length category value for the record history length category of the target individual may be determined based on a measure of length of the available credit history of the target individual within the last years. In some of the noted exemplary embodiments: (i) if the measure of length of the available credit history of the target individual falls below a first threshold (e.g., 12 months), the record history length category value for record history length category of the target individual may be assigned a lowest value (e.g., a value of 12); (ii) if the measure of length of the available credit history of the target individual falls more than or equal to the first threshold but less than a second threshold (e.g., 24 months), the record history length category value for record history length category of the target individual may be assigned a second lowest value (e.g., a value of 35); (iii) if the measure of length of the available credit history of the target individual falls more than or equal to the second threshold but less than a third threshold (e.g., 47 months), the record history length category value for record history length category of the target individual may be assigned a third lowest value (e.g., a value of 60); and (iv) if the measure of length of the available credit history of the target individual falls more than or equal to the third threshold, the record history length category value for record history length category of the target individual may be assigned a fourth lowest value (e.g., a value of 75). In some embodiments, the noted record length history category can be mapped to a target condition onset delay category as part of generating a cross-domain mapping for the credit risk modeling with respect to a polygenic risk scoring predictive domain.

In some embodiments, a target condition onset delay category describes an inferred risk category that relates to a magnitude of the temporal interval between an estimated onset point in time for a corresponding target condition in a target individual and a current individual. The target condition onset delay category value for the target condition onset delay category may be determined based on a length of time related to management of the corresponding target condition (e.g., a corresponding disease, a corresponding phenotype, and/or the like). In some embodiments, the target condition onset delay category value for the target condition onset delay category may be determined using a GLM that is configured to generate positive values.

At step/operation 504, the predictive data analysis computing entity 106 maps a record diversity category of the initial risk scoring model to a current therapeutic management category. For example, the predictive data analysis computing entity 106 may map a credit mix category associated with a credit risk scoring model to a current therapeutic management category. Aspects of record diversity categories and current therapeutic management categories are described in greater detail below.

In some embodiments, a record diversity category describes an initial risk category for an initial risk scoring model that represents a property related to a number of record sources associated an activity record utilized by the initial risk scoring model to generate initial risk predictions. For example, if the initial risk scoring model is a credit risk scoring model, the record diversity category value for the record diversity category may describe a number of bankcard trade lines associated with a corresponding credit history during a current time and/or during a particular historical timeframe. In some of the noted exemplary embodiments: (i) if the number of bankcard trade lines is less than a first threshold (e.g., one), the record diversity category value for the record diversity category may be assigned a lowest value (e.g., a value of 15); (ii) if the number of bankcard trade lines is more than or equal to the first threshold but less than a second threshold (e.g., two), the record diversity category value for the record diversity category may be assigned a second lowest value (e.g., a value of 25); (iii) if the number of bankcard trade lines is more than or equal to the second threshold but less than or equal to a third threshold (e.g., three), the record diversity category value for the record diversity category may be assigned a third lowest value (e.g., a value of 50); (iv) if the number of bankcard trade lines is more than or equal to the third threshold but less than a fourth threshold (e.g., four), the record diversity category value for the record diversity category may be assigned a fourth lowest value (e.g., a value of 60); and (v) if the number of bankcard trade lines during the last six months is more than or equal to the fourth threshold, the record diversity category value for the record diversity category may be assigned a fifth lowest value (e.g., a value of 50). In some embodiments, the record diversity category can be mapped to a current therapeutic management category as part of generating a cross-domain mapping for the credit risk modeling with respect to a polygenic risk scoring predictive domain.

In some embodiments, a current therapeutic management category describes an inferred risk category that relates to a current therapeutic approach to a target condition of a target individual. For example, the current therapeutic management category may relate to a current disease management and/or a current medication adherence of a target individual with respect to a target condition. In some embodiments, the current therapeutic management category value for the current therapeutic management category is determined based on at least one of the following: (i) the polychronic diseases present in the target individual and their associated comorbidity in relation to the target condition, (ii) a measure of wellness/lifestyle of the target individual, and (iii) a measure of adherence of the target individual to medical and/or pharmaceutical guidelines for prevention and/or treatment of the target condition. In some embodiments, the current therapeutic management category value for the current therapeutic management category is determined using a GLM. In some embodiments, at least a portion of the data used to determine the current therapeutic management category value for the current therapeutic management category is generated using a non-linear prediction model, such as non-linear RX adherence prediction machine learning model.

At step/operation 505, the predictive data analysis computing entity 106 maps a query frequency category of the initial risk scoring model to a genetic variance category. For example, the predictive data analysis computing entity 106 may map a new credit inquiry recency category associated with a credit risk scoring model to a genetic variance category. Aspects of query frequency categories and genetic variance categories are described in greater detail below.

In some embodiments, a query frequency category describes an initial risk category for an initial risk scoring model that represents a property related to a recency of obtaining an initial risk prediction by the initial risk scoring model and/or to frequency of obtaining an initial risk prediction by the initial risk scoring model within a particular historical timeframe (e.g., within the last six months). For example, if the initial risk scoring model is a credit risk scoring model, the query frequency category value for the query frequency category may describe the number of credit inquiries performed using the credit risk scoring model during the last six months. In some of the noted exemplary embodiments: (i) if the number of the new credit inquiries during the last six months is less than a first threshold (e.g., one), the query frequency category value for the query frequency category may be assigned a highest value (e.g., a value of 70); (ii) if the number of the new credit inquiries during the last six months is more than or equal to the first threshold but less than a second threshold (e.g., two), the query frequency category value for the query frequency category may be assigned a second highest value (e.g., a value of 60); (iii) if the number of the new credit inquiries during the last six months is more than or equal to the second threshold but less than or equal to a third threshold (e.g., three), the query frequency category value for the query frequency category may be assigned a third highest value (e.g., a value of 45); (iv) if the number of the new credit inquiries during the last six months is more than or equal to the third threshold but less than a fourth threshold (e.g., four), the query frequency category value for the query frequency category may be assigned a fourth highest value (e.g., a value of 25); and (v) if the number of the new credit inquiries during the last six months is more than or equal to the fourth threshold, the query frequency category value for the query frequency category may be assigned a fifth highest value (e.g., a value of 20). In some embodiments, the query frequency category can be mapped to a genetic variance category as part of generating a cross-domain mapping for the credit risk modeling with respect to a polygenic risk scoring predictive domain.

In some embodiments, a genetic variance category describes an inferred risk category that relates to a variation of at least a portion of a genetic composition of a target individual relative to genetic population of an observed population and/or relative a current human genome reference. In some of the noted embodiments, the genetic variance category value for the genetic variance category is determined based on at least one of: (i) the number of genetic and/or medical tests performed during a historical timeframe, (ii) the identity of panels screened during the noted genetic and/or medical texts, and (iii) any VUSs found during the noted genetic and/or medical texts. In some embodiments, the genetic variance category value for the genetic variance category is determined using a GLM. In some embodiments, the genetic variance category value for the genetic variance category is determined using a non-linear prediction model. In some embodiments, the genetic variance category value for the genetic variance category is determined using a VUS probability distribution, such as a VUS probability that relates clinical significance of particular VUSs with respect to particular target conditions.

Returning to FIG. 4, at step/operation 402, the predictive data analysis computing entity 106 determines an inferred risk category value for each inferred risk category that is mapped to an initial risk category of the initial risk model by the cross-domain mapping, where determining the inferred risk category value for an inferred risk category is performed based on the observed input variables for the inferred risk category. An observed input variable may be any data object that is used to determine an inferred risk category value. Selection of the observed input variables for each inferred risk category value may be performed in a manner that is configured to facilitate adoption of a resulting inferred risk category value within a computational structure of the initial risk scoring model (i.e., the model that is eventually modified to perform health-related predictive data analysis, as described in greater detail below in relation to steps/operations 403-404).

In some embodiments, an inferred risk category value may be a data object that describes a singular value and/or a singular vector that contains information related to a corresponding inferred risk category configured to be transferred as inputs to an initial risk scoring model. Accordingly, the inferred risk category value is a mapping of selected information from a secondary predictive domain other than the default predictive domain of the initial risk scoring model (e.g., from the polygenic risk scoring predictive domain, which may be distinct from the predictive domain of an initial risk scoring model) to a variable of the initial risk scoring model. For example, given a medical history category as an inferred risk category, the inferred risk category value for the noted medical history category may describe the sets of medical history events that are encoded into a common representation (e.g., into a common scalar representation) in order to input to an initial risk scoring (e.g., to a credit risk scoring model).

As noted above, an inferred risk category value may be determined based on observed input values that are deemed related to the inferred risk category of the inferred risk category value. In some of the noted embodiments, generating an inferred risk category value for an inferred risk category comprises processing the one or more observed input variables associated with the inferred risk category using a trained machine learning model associated with the inferred risk category to generate the inferred risk category value.

For example, a medical history category value for a medical history category may be determined based on at least one of medical symptom history (e.g., data about severity of medical symptoms of the target individual over the particular historical timeframe), genetic variation data (e.g., data about SNPs, CNVs, indels, gene fusions, duplications, and/or other genetic variations that are present in the genome of the target individual), and/or the like. In some embodiments, a medical history category value for the medical history category may be determined based on at least one of the following: a machine learning model (such as a trained GLM) that is configured to process the medical symptom history data associated with the target individual in order to generate a medical symptom history representation for the target individual, and a non-linear predictive model that is configured to process the genetic variation data (e.g., the CNV data) associated with the target individual in order to generate a genetic variation representation for the target individual.

As another example, a current phenotype category value for a current phenotype category may be determined based on a measure of current genomic utilization of a target individual. In some embodiments, the current phenotype category value for the current phenotype category is determined using a GLM model. In some embodiments, the current phenotype category value for the current phenotype category is determined using a non-linear predictive model, such as a Bell curve regression model.

As yet another example, a target condition onset delay category value for a target condition onset delay category may be determined based on a length of time related to management of the corresponding target condition (e.g., a corresponding disease, a corresponding phenotype, and/or the like). In some embodiments, the target condition onset delay category value for the target condition onset delay category may be determined using a GLM that is configured to generate positive values.

As a further example, a current therapeutic management category value for a current therapeutic management category is determined based on at least one of the following: (i) the polychronic diseases present in the target individual and their associated comorbidity in relation to the target condition, (ii) a measure of wellness/lifestyle of the target individual, and (iii) a measure of adherence of the target individual to medical and/or pharmaceutical guidelines for prevention and/or treatment of the target condition. In some embodiments, the current therapeutic management category value for the current therapeutic management category is determined using a GLM. In some embodiments, at least a portion of the data used to determine the current therapeutic management category value for the current therapeutic management category is generated using a non-linear prediction model, such as non-linear RX adherence prediction machine learning model.

As an additional example, a genetic variance category value for a genetic variance category is determined based on at least one of: (i) the number of genetic and/or medical tests performed during a historical timeframe, (ii) the identity of panels screened during the noted genetic and/or medical texts, and (iii) any VUSs found during the noted genetic and/or medical texts. In some embodiments, the genetic variance category value for the genetic variance category is determined using a GLM. In some embodiments, the genetic variance category value for the genetic variance category is determined using a non-linear prediction model. In some embodiments, the genetic variance category value for the genetic variance category is determined using a VUS probability distribution, such as a VUS probability that relates clinical significance of particular VUSs with respect to particular target conditions.

At step/operation 403, the predictive data analysis computing entity 106 determines a per-category weight value for each inferred risk category that is mapped to an initial risk category of the initial risk model by the cross-domain mapping. Aspects of per-category weight values and exemplary embodiments for generating the noted per-category weight values are described in greater detail below.

In some embodiments, a per-category weight value describes an estimated significance of a corresponding inferred risk category value for a corresponding inferred risk category to determining a health-related risk prediction for a target individual with respect to a target condition. In some of the noted embodiments, the per-category weight values provide a technique through which developers of health-related predictive data analysis models can transfer domain-level information about relationships between observed variables and target conditions to domain-agnostic and/or domain-alien initial risk scoring models, such as credit risk scoring models in relation to health-related predictive data analysis models. For example, the medical history category value for the medical history category may be deemed more pertinent for a first target condition (e.g., diabetes) relative to a second target condition (e.g., AIDS). In the noted example, the per-category weight value for the medical history category relative to the first target condition will likely be higher than the per-category weight value for the medical history category relative the second target condition. As another example, the genetic variation category value for the genetic variation category may be deemed more pertinent for a first target condition (e.g., hemophilia) relative to a second target condition (e.g., common cold). In the noted example, the per-category weight value for the genetic variation category relative to the first target condition will likely be higher than the per-category weight value for the genetic variation category relative the second target condition.

In some embodiments, each per-category weight value for an inferred risk category is determined in accordance with an optimization-based training technique and based on ground-truth health-related risk predictions for a group of training individual-condition pairs. In some of the noted embodiments, the predictive data analysis computing entity 106 processes (e.g., using a machine learning framework, such as a neural network model) each inferred risk category value for an inferred risk category that is associated with a particular ground-truth polygenic prediction of the ground-truth health-related risk predictions in accordance with initial per-category weight values for the inferred risk categories to determine an inferred health-related risk prediction for the particular ground-truth polygenic prediction. Afterward, the predictive data analysis computing entity 106 generates a utility model (e.g., a loss model, a reward model, and/or the like) based on a measure of deviation between each ground-truth polygenic prediction and the corresponding inferred health-related risk prediction for the ground-truth polygenic prediction. Thereafter, the predictive data analysis computing entity 106 optimizes (e.g., minimizes a loss model, maximizes a reward model, and/or the like) the measure of deviation and adopts the per-category weight values that optimize the measure of deviation as the final per-category weight values for the inferred risk categories. In some of the noted embodiments, the noted optimization may be performed using an optimization-based training technique, such as using gradient descent and/or gradient descent with backpropagation. In some embodiments, the initial risk scoring model defines an initial weight for each initial risk category, and each initial per-category weight value for an inferred risk category is determined based on the initial weight value for the initial risk category that is mapped to the inferred risk category according to the cross-domain mapping.

In some embodiments, the initial risk scoring model defines an initial weight for each initial risk category, and each final per-category weight value for an inferred risk category is determined based on the initial weight value for the initial risk category that is mapped to the inferred risk category according to the cross-domain mapping. Thus, the predictive data analysis computing entity 106 may in some embodiments adopt the weight values specified by the initial risk scoring model as the final weight values for inferred risk categories.

At step/operation 404, the predictive data analysis computing entity 106 generates a health-related risk prediction by processing each inferred risk category value for an inferred risk category and each per-category weight value for an inferred risk category value. In some embodiments, the predictive data analysis computing entity 106 generates a weighted risk category value for each inferred risk category by applying (e.g., multiplying) the per-category weight value for the inferred risk category value to the inferred risk category value for the inferred risk category.

In some embodiments, generating the health-related risk prediction is performed using the below Equation 1:

$$p = \frac{exp ( {β}_{0} + {β}_{1} \cdot {x}_{1} + … + {β}_{n} \cdot {x}_{n} )}{1 + exp ( {β}_{0} + {β}_{1} \cdot {x}_{1} + … + {β}_{n} \cdot {x}_{n} )} \qquad Equation 1$$

In Equation 1: (i) p is the health-related risk prediction, (ii) each xᵢ is an inferred risk category value for an inferred risk category i, (iii) each βᵢ is the per-category weight value for an inferred risk category i, (iv) xᵢ βᵢ is the weighted risk category value for an inferred risk category i, and (v) n is the number of inferred risk categories (which may be equivalent to the number of initial risk categories). In some embodiments, each per-category weight value is determined using the Equation 1 and by applying an optimization technique that is in accordance with a maximum likelihood estimation.

In some embodiments, the predictive data analysis computing entity 106 combines the health-related risk prediction with a PRS after calculation of the PRS. This combination may be performed using a trained GLM and/or using a trained ensemble machine learning model. The output of the combination may then be adopted as the updated health-related risk prediction. In some embodiments, the output of the noted combination may be adopted as the updated health-related risk prediction if it generates a desired level of accuracy when tested in relation to labeled validation data.

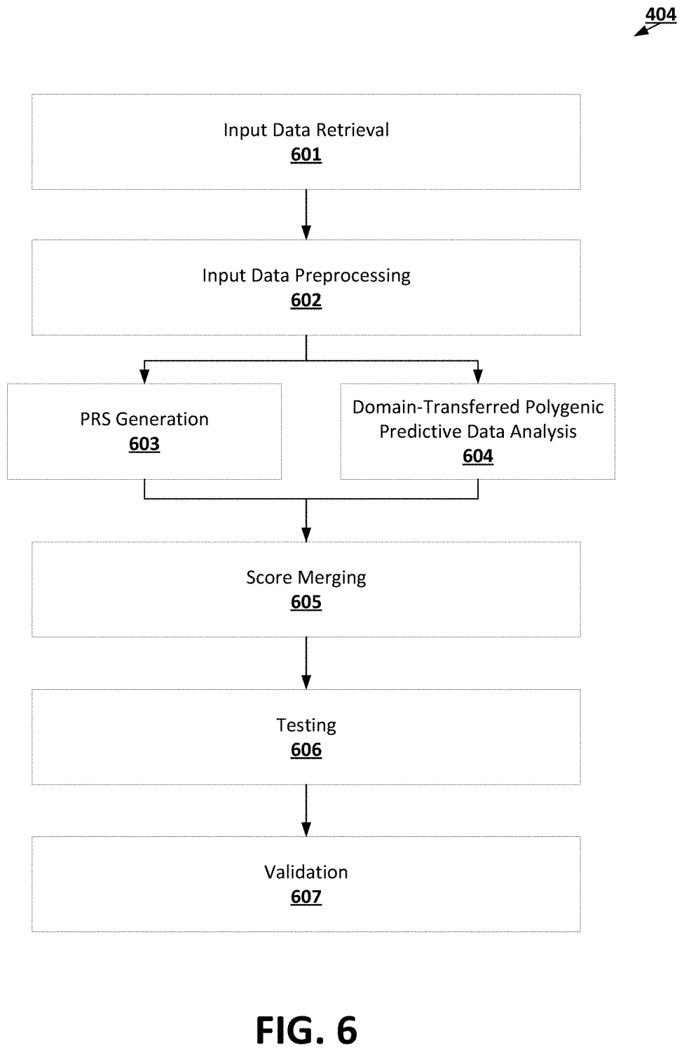

In some embodiments, step/operation 404 may be performed in accordance with the process depicted in FIG. 6. As depicted in FIG. 6, the predictive data analysis computing entity 106 first performs input data retrieval 601, which may include retrieving base data (e.g., summary statistics, betas, odds ratios, and/or the like) as well as target data (e.g., individual-level genotype and phenotype data). Afterward, the predictive data analysis computing entity 106 performs input data preprocessing 602, which may include performing quality control (e.g., performed using a Graphical Analysis Workstation (GAWS), performed using sample overlap techniques, performed using relatedness techniques, performed using population structure techniques, and/or the like). A purpose of the input data preprocessing 602 may be to retrain sets of SNPs that overlap between SNP and target data.

Next, the predictive data analysis computing entity 106 performs PRS generation 603 (e.g. using at least one of linkage disequilibrium (LD) adjustment such as via clumping, Beta shrinkage such as via least absolute shrinkage and selection operator (LASSO) and/or via Ridge regression, and P-value thresholding via one or more threshold P values). Moreover, the predictive data analysis computing entity 106 performs domain-transferred health-related predictive data analysis 604 using at least some of the techniques described above with reference to FIGS. 4-5. Thereafter, the predictive data analysis computing entity 106 performs score merging 605 by merging the PRS and the polygenic risk score generated at the domain-transferred health-related predictive data analysis 604. Subsequently, the predictive data analysis computing entity 106 performs testing 606 (e.g., association testing, out-of-sample testing, and/or the like) of the merged output. Finally, the predictive data analysis computing entity 106 proceeds to perform validation 607 (e.g., using K-fold cross-validation) of the merged output based on the results of the testing 606.

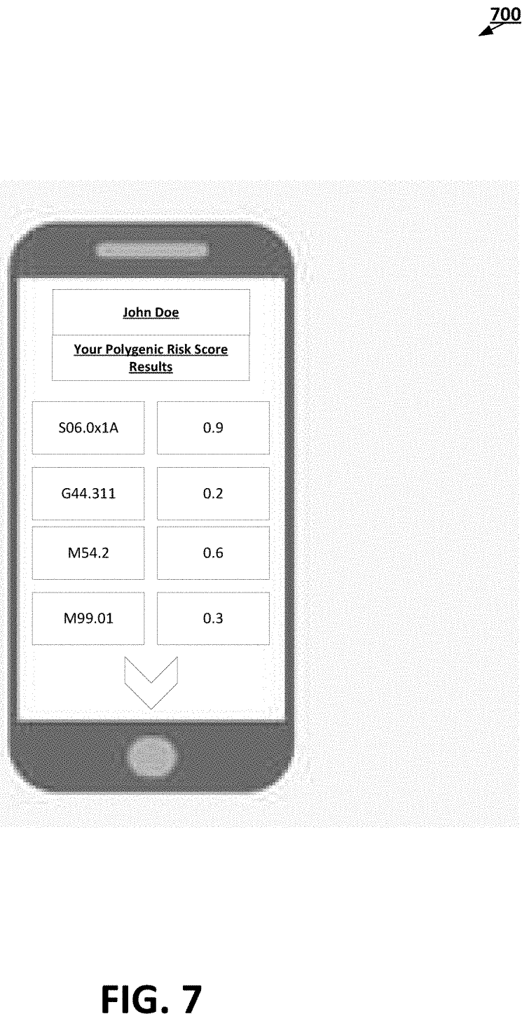

At step/operation 405, the predictive data analysis computing entity 106 performs one or more prediction-based actions based on the health-related risk prediction. Examples of prediction-based actions including displaying a user interface that displays health-related risk predictions for a target individual with respect to a set of conditions. For example, as depicted in FIG. 7, the predictive output user interface 700 depicts the health-related risk prediction for a target individual with respect to four target conditions each identified by the International Statistical Classification of Diseases and Related Health Problems (ICD) code of the noted four target conditions.

For example, the predictive output user interface 700 of FIG. 7 depicts that the target individual has a health-related risk prediction of 0.9 with respect to the condition with the ICD code S06.0x1A, a health-related risk prediction of 0.2 with respect to the condition with the ICD code G44.311, a health-related risk prediction of 0.6 with respect to the condition with the ICD code M54.2, and a health-related risk prediction of 0.3 with respect to the condition with the ICD code M99.01.

In some embodiments, the predictive data analysis computing entity 106 may determine one or more patient health predictions (e.g., one or more urgent care predictions, one or more medication need predictions, one or more visitation need predictions, and/or the like) based on the health-related risk prediction and perform one or more prediction-based actions based on the noted determined patient health predictions. Examples of prediction-based actions that may be performed based on the patient health predictions include automated physician notifications, automated patient notifications, automated medical appointment scheduling, automated drug prescription recommendation, automated drug prescription generation, automated implementation of precautionary actions, automated hospital preparation actions, automated insurance workforce management operational management actions, automated insurance server load balancing actions, automated call center preparation actions, automated hospital preparation actions, automated insurance plan pricing actions, automated insurance plan update actions, and/or the like.

### VI. CONCLUSION

Many modifications and other embodiments will come to mind to one skilled in the art to which this disclosure pertains having the benefit of the teachings presented in the foregoing descriptions and the associated drawings. Therefore, it is to be understood that the disclosure is not to be limited to the specific embodiments disclosed and that modifications and other embodiments are intended to be included within the scope of the appended claims. Although specific terms are employed herein, they are used in a generic and descriptive sense only and not for purposes of limitation.

1. A computer-implemented method for performing health-related predictive data analysis for a target individual with respect to a target condition, the computer-implemented method comprising:
identifying an initial risk scoring model, wherein the initial risk scoring model is associated with a plurality of initial risk categories;
generating a cross-domain mapping of the initial risk scoring model, wherein: (i) the cross-domain mapping maps each initial risk category of the plurality of initial risk categories to an inferred risk category of a plurality of inferred risk categories, and (ii) each inferred risk category of the plurality of inferred risk categories is associated with one or more observed input variables for the target individual;
for each inferred risk category of the plurality of inferred risk categories:
determining an inferred risk category value for the inferred risk category based on the one or more observed input variables for the inferred risk category,
determining a per-category weight value for the inferred risk category value, and
determining a weighted risk category value for the inferred risk category based on the inferred risk category value for the inferred risk category and the per-category weight value for the inferred risk category;
processing each weighted risk category value for an inferred risk category of the plurality of inferred risk categories using the initial risk scoring model and in accordance with the cross-domain mapping in order to generate a health-related risk prediction for the target individual with respect to the target condition; and
performing one or more prediction-based actions based on the health-related risk prediction.

2. The computer-implemented method of claim 1, wherein:
the plurality of initial risk categories comprise a compliance history category,
the plurality of inferred risk categories comprise a medical history category, and
the cross-domain mapping maps the compliance history category to the medical history category.

3. The computer-implemented method of claim 1, wherein:
the plurality of initial risk categories comprise a record magnitude category,
the plurality of inferred risk categories comprise a current phenotype category, and
the cross-domain mapping maps the record magnitude category to the current phenotype category.

4. The computer-implemented method of claim 1, wherein:
the plurality of initial risk categories comprise a record history length category,
the plurality of inferred risk categories comprise a target condition onset delay category, and
the cross-domain mapping maps the record history length to the target condition onset delay category.

5. The computer-implemented method of claim 1, wherein:
the plurality of initial risk categories comprise a record diversity category,
the plurality of inferred risk categories comprise a current therapeutic management category, and
the cross-domain mapping maps the record diversity category to current therapeutic management category.

6. The computer-implemented method of claim 1, wherein:
the plurality of initial risk categories comprise a query frequency category,
the plurality of inferred risk categories comprise a genetic variance category, and
the cross-domain mapping maps the query frequency category to current genetic variance category.

7. The computer-implemented method of claim 1, wherein generating each inferred risk category value for an inferred risk category of the plurality of inferred risk categories comprises:
processing the one or more observed input variables associated with the inferred risk category using a trained machine learning model associated with the inferred risk category to generate the inferred risk category value.

8. The computer-implemented method of claim 1, wherein:
the initial risk scoring model defines an initial weight for each initial risk category of the plurality of initial risk categories, and
each per-category weight value for an inferred risk category of the plurality of inferred risk categories is determined based on the initial weight value for the initial risk category that is mapped to the inferred risk category according to the cross-domain mapping.

9. The computer-implemented method of claim 1, wherein each per-category weight value for an inferred risk category of the plurality of inferred risk categories is determined in accordance with an optimization-based training technique and based on ground-truth health-related risk predictions for a group of training individual-condition pairs.

10. The computer-implemented method of claim 1, wherein the health-related risk prediction is updated in accordance with a Polygenic Risk Score (PRS) for the target individual with respect to the target condition.

11. An apparatus for performing health-related predictive data analysis for a target individual with respect to a target condition, the apparatus comprising at least one processor and at least one memory including program code, the at least one memory and the program code configured to, with the processor, cause the apparatus to at least:
identify an initial risk scoring model, wherein the initial risk scoring model is associated with a plurality of initial risk categories;
generate a cross-domain mapping of the initial risk scoring model, wherein: (i) the cross-domain mapping maps each initial risk category of the plurality of initial risk categories to an inferred risk category of a plurality of inferred risk categories, and (ii) each inferred risk category of the plurality of inferred risk categories is associated with one or more observed input variables for the target individual;
for each inferred risk category of the plurality of inferred risk categories:
determine an inferred risk category value for the inferred risk category based on the one or more observed input variables for the inferred risk category,
determine a per-category weight value for the inferred risk category value, and
determine a weighted risk category value for the inferred risk category based on the inferred risk category value for the inferred risk category and the per-category weight value for the inferred risk category;
process each weighted risk category value for an inferred risk category of the plurality of inferred risk categories using the initial risk scoring model and in accordance with the cross-domain mapping in order to generate a health-related risk prediction for the target individual with respect to the target condition; and
perform one or more prediction-based actions based on the health-related risk prediction.

12. The apparatus of claim 11, wherein:
the plurality of initial risk categories comprise a compliance history category,
the plurality of inferred risk categories comprise a medical history category, and
the cross-domain mapping maps the compliance history category to the medical history category.

13. The apparatus of claim 11, wherein:
the plurality of initial risk categories comprise a record magnitude category,
the plurality of inferred risk categories comprise a current phenotype category, and
the cross-domain mapping maps the record magnitude category to the current phenotype category.

14. The apparatus of claim 11, wherein:
the plurality of initial risk categories comprise a record history length category,
the plurality of inferred risk categories comprise a target condition onset delay category, and
the cross-domain mapping maps the record history length to the target condition onset delay category.

15. The apparatus of claim 11, wherein:
the plurality of initial risk categories comprise a record diversity category,
the plurality of inferred risk categories comprise a current therapeutic management category, and
the cross-domain mapping maps the record diversity category to current therapeutic management category.

16. A computer program product for performing health-related predictive data analysis for a target individual with respect to a target condition, the computer program product comprising at least one non-transitory computer-readable storage medium having computer-readable program code portions stored therein, the computer-readable program code portions configured to:
identify an initial risk scoring model, wherein the initial risk scoring model is associated with a plurality of initial risk categories;
generate a cross-domain mapping of the initial risk scoring model, wherein: (i) the cross-domain mapping maps each initial risk category of the plurality of initial risk categories to an inferred risk category of a plurality of inferred risk categories, and (ii) each inferred risk category of the plurality of inferred risk categories is associated with one or more observed input variables for the target individual;
for each inferred risk category of the plurality of inferred risk categories:
determine an inferred risk category value for the inferred risk category based on the one or more observed input variables for the inferred risk category,
determine a per-category weight value for the inferred risk category value, and
determine a weighted risk category value for the inferred risk category based on the inferred risk category value for the inferred risk category and the per-category weight value for the inferred risk category;
process each weighted risk category value for an inferred risk category of the plurality of inferred risk categories using the initial risk scoring model and in accordance with the cross-domain mapping in order to generate a health-related risk prediction for the target individual with respect to the target condition; and
perform one or more prediction-based actions based on the health-related risk prediction.

17. The computer program product of claim 16, wherein:
the plurality of initial risk categories comprise a compliance history category,
the plurality of inferred risk categories comprise a medical history category, and
the cross-domain mapping maps the compliance history category to the medical history category.

18. The computer program product of claim 16, the plurality of initial risk categories comprise a record magnitude category,
the plurality of inferred risk categories comprise a current phenotype category, and
the cross-domain mapping maps the record magnitude category to the current phenotype category.

19. The computer program product of claim 16, wherein:
the plurality of initial risk categories comprise a record history length category,
the plurality of inferred risk categories comprise a target condition onset delay category, and
the cross-domain mapping maps the record history length to the target condition onset delay category.

20. The computer program product of claim 16, wherein:
the plurality of initial risk categories comprise a record diversity category,
the plurality of inferred risk categories comprise a current therapeutic management category, and
the cross-domain mapping maps the record diversity category to current therapeutic management category.
